(19) United States
(12) Patent Application Publication
(10) Pub. No.: US 2005/0143736 A1
(43) Pub. Date: Jun. 30, 2005

(54) Compression plate for bone fixation

(76) Inventor: Eduardo da Frota Carrera, Sao Paulo-SP (BR)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 10/960,470

(22) Filed: Oct. 7, 2004

(30) Foreign Application Priority Data
Oct. 8, 2003 (BR) PI 0304144-1

(51) Int. Cl. A61F 2/30

(52) U.S. Cl. 606/60

(57) ABSTRACT

The present invention refers to fixing plates to be applied on bones and, more specifically, to a plate which, besides fixing, promotes compression with the purpose to increase the stability of the fractured bone set or bones suffering osteotomy.

### BACKGROUND OF THE INVENTION

The present invention refers to fixing plates to be applied to bones and, more specifically, to a plate which, besides fixing, also promotes compression with the purpose to increase the stability of the set.

The main object of the present invention is to fix broken bones or those, which suffered osteotomy (i. e. bones which are sawed to be repositioned for the correction of some existing deformation).

The current treatment of fractures aims, among others, to fix displaced fragments and make them stable. The object is therefore to find a material promoting the best possible fixing and stabilization of fractured fragments. Currently used plates to treat fractures promote good fixing and stabilization, but can fail, depending on the fracture.

### SUMMARY OF THE INVENTION

The innovation as presented herein refers to the concept of fixing by using an angled blade plate similar to the conventional one, but with two special orifices through which a special screw passes which, when screwed, will determine compression on the blade and the latter, on the other hand, will suffer flexure on the blade. This device generates compression between fractured fragments, which is desirable to obtain better fixing and stabilization of the fracture or osteotomy.

The plate is introduced into the human bones, between the two or more fractured segments, to promote fixing and stabilization of these fragments.

The present invention is constituted by an angled metal blade plate (with varied angles for each kind of fracture—initially plates with 80, 90 and 1000 angles) used to fix bone segments. The material has the shape of an inverted “L”.

The side metal surface with five orifices is called and considered as a fixing “plate”, the upper metal surface with one orifice is called and considered as “blade”. Such blade plate fixes two or more fractured bone segments to allow to appropriately fracture healing. Besides fixing the fracture, the plate allows compression on the focus of the fracture, by means of an “interference screw” passed from the “plate” to the “blade”.

This kind of fixing has shown more resistance over other already existent types of blade plates, according to biomechanical tests. Furthermore, better fixing of fractured or osteotomy segments, over the fixing of the fracture or osteotomy with already existent conventional plates, was also reached.

### BRIEF DESCRIPTION OF THE DRAWINGS

The present invention will be better understood by examining the attached figures, given merely as examples, but not limiting, in which:

FIG. 1 is a side view of the plate;

FIG. 2 is a front view of the plate;

FIG. 3 is an upper view of the plate;

FIG. 4 is a side view of the screw to be applied to the plate;

FIG. 5 is a front view of the screw head to be applied to the plate;

FIG. 6 is an upper view of the plate and screw set;

FIG. 7 is a side view of the plate and screw set;

FIG. 8 is a view of the plate as applied to a humerus, including an X-ray view;

FIG. 9 is a side view of a plate set with various inclination degrees;

FIG. 10 is an end view of a plate set with various inclination degrees;

FIG. 11 is a view of the model as used to simulate efforts over the plate; and

FIG. 12 is a graph relating force and maximum displacement suffered by the plate.

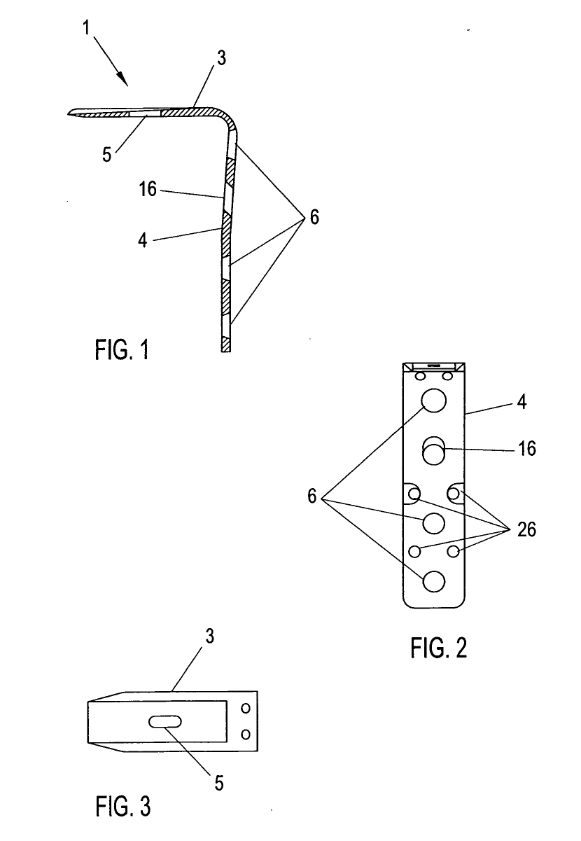

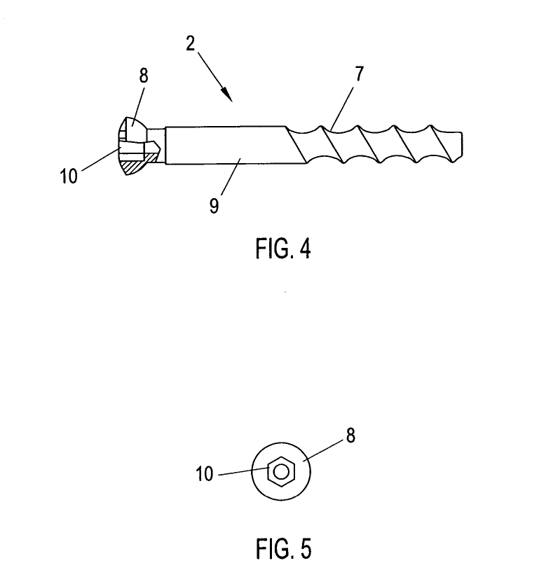

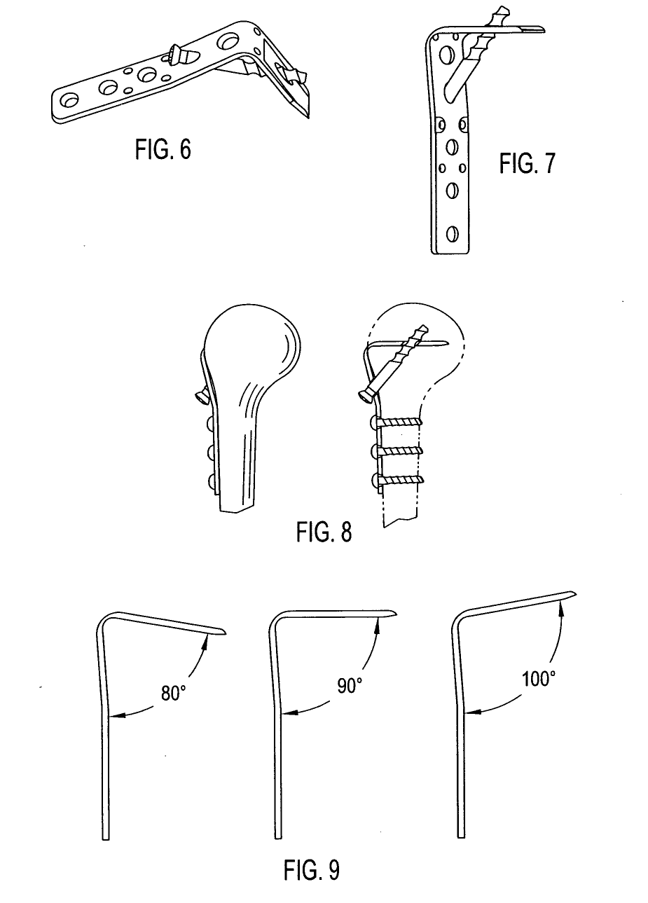

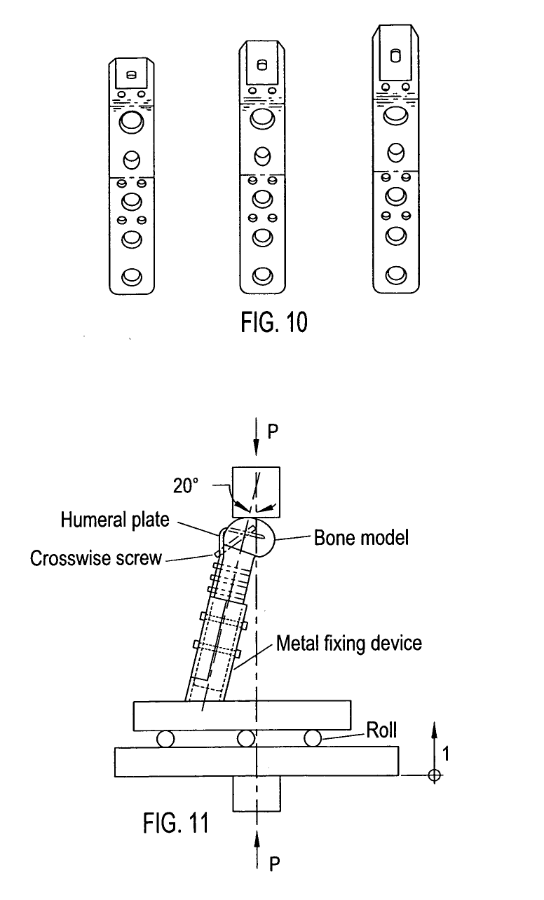

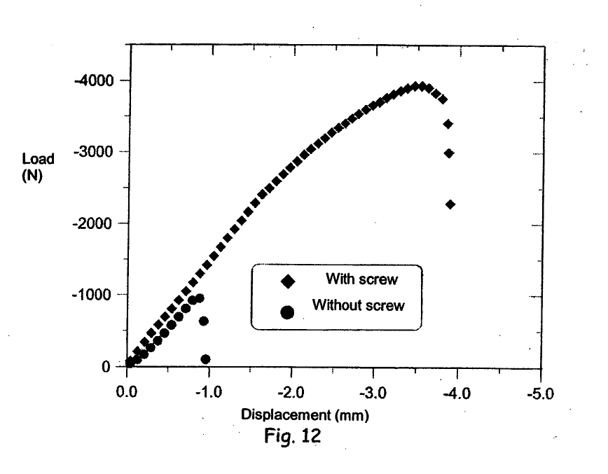

### DESCRIPTION OF THE PREFERRED EMBODIMENT

The present invention is constituted by a set of plate (1) and “interference” screw (2), being said screw (2) fixed to the plate (1).

The plate (1) is constituted by a plate (4) having holes (6) for cortical and/or spongeous bone screws, and a special oblong hole (16) for the “interference” screw (2).

There are also four small orifices (26) located in parallel on the plate (4), to which a guide for introduction of the “interference” screw (2) is coupled.

The “blade” (3) corresponding to the higher part of the material has an oblong hole (5).

The screw (2) has a screw (7), a head (8) and a plain region (9). The screw (7) path is different to allow the screw (2) to be fitted between the plate (4) and the blade (3) of the plate (1). The screw (2) has a circular head (8) and is provided with a recess (10) to fit a screwdriver; said recess may be in the format of a gap, Philips, Allen or torque, preferably Allen.

We should consider that the length of the plate segment called “blade” (3) and the length of the segment called “plate” (4) with the orifices are variable. Furthermore, the angle formed between the segment called “blade” and “plate” is also variable.

The length of these segments and the angles change with the type of fracture or osteotomy and the fixing requirements.

The “plate” (4) may be longer or shorter, as well as the blade (3). The segment called “plate” (4) may have as many orifices (6) as required, depending on the length of the plate.

The method to fix the plate to the indicated place is simple and constitutes the preparation of the higher part of the bone to be corrected from an opening to fit the blade (3) of the plate (1). Subsequently, holes to fix conventional screws passing through the holes (6) of the plate (1) are made alongside the bone. Then, an inclined hole is made, allowing the passage of the screw (2) through the hole (16) of the plate (4) and allowing it to be screwed until reaching the oblong hole (5) of the blade (3).

Upon reaching the oblong hole (5), as a function of the screw (7), the screw (2) starts to traction the blade (3) of the plate (1), making it “compress” the region by means of flexure of the plate (4) over the “blade” (3), therefore promoting compression on the region to be consolidated and allowing faster recovery of the patient.

Tests were made to evaluate the efficacy of the present invention.

a) Received Materials:

Three models of Angled Humeral Plates were received for the static flexure assays, with angles of 80°, 90° and 100°. The plates were manufactured with austenitic stainless steel ASTM F138. A total of 25 plates were received for assays, being five 80° plates, 15×90° plates and five 100° plates.

According to the model as presented on FIG. 11, tests were made to verify the flexure-resistant values, as per the tables below.

Values of Standards as Obtained from Static Flexure Assays on Angled Humeral Plates (90°) Fixed to the Bone Model with no Crossed Screw

| | Resistance to | |
|---|---|---|
| Sample | Flexure (N) | Rigidity (N/mm) |
| 1 | −274 | −0.3 |
| 2 | −983 | −0.7 |
| 3 | −301 | −0.4 |
| 4 | −963 | −0.6 |
| 5 | −483 | −0.5 |

Result average was −601 N with standard deviation of 349 N and rigidity was 0.5 N/mm, with standard deviation of 0.2 N/mm.

Values of Standards as Obtained from Static Flexure Assays on Angled Humeral Plates (90°) Fixed to the Bone Model with Crossed Screw

| | Resistance to | |
|---|---|---|
| Sample | Flexure (N) | Rigidity (N/mm) |
| 1 | −4173 | −3.6 |
| 2 | −4140 | −5.0 |
| 3 | −4010 | −3.3 |
| 4 | −3770 | −4.3 |
| 5 | −3930 | −3.5 |

Result average was −4005 N with standard deviation of 164 N and rigidity was 3.9 N/mm with standard deviation of 0.7 N/mm.

As we can see, the result obtained with the plate without the “interference” screw (2) (601±349 N) is much lower than the one obtained with such screw (2) (4.005±164 N) over the resistance to flexure force (the angled plate without “interference” screw (2) is similar to angled plates already available in the market). This is the force suffered by the bone at the fractured place. The higher the resistance against these forces, the better will be the fixing and the better the fixing material for use in fracture treatment will be considered.

The advantage of the present invention over existing blade plates is the “interference” screw (2) and the flexure suffered by the “blade” (3) of the plate (1), promoting more stable fixing of the set (plate and fractured bone) when the “interference” screw (2) is tightened.

1. Compression plate to fix bones constituted by a plate (4) and blade (3) approximately crosswise to the plate (4) with holes (6) and orifices (26), in which the plate (4) and blade (3) are provided with oblong holes (5) and (16) to introduce and fix the screw (2) provided with specific screw (7) which path allows to lock and traction between the plate (4) and the blade (3).

2. Fixing screw for the compression plate of claim 1, which has a head (8), plain region (9) and screw with specific path (7).

3. Screw of claim 2, which has on its circular head (8) a recess (10), which may be in a gap, Philips, Allen or torque format, preferably Allen format.

4. Set to fix bones which is formed by the plate (1) and the screw (2).
